(19) United States
(12) Patent Application Publication
(10) Pub. No.: US 2026/0091190 A1
(43) Pub. Date: Apr. 2, 2026

(54) TRACHEOSTOMY TUBE STRUCTURE

(71) Applicant: Taipei Medical University, Taipei City (TW)

(72) Inventors: Pai-Chien Chou, Taipei City (TW); Chien-Hua Chen, Taipei City (TW); Hsueh-Lien Su, Taipei City (TW); Ya-Ting Juang, Taipei City (TW)

(21) Appl. No.: 19/341,275

(22) Filed: Sep. 26, 2025

(30) Foreign Application Priority Data
Sep. 27, 2024 (TW) 113136834

(51) Int. Cl. A61M 16/04

(52) U.S. Cl. CPC A61M 16/0465; A61M 16/0402; A61M 2205/0216; A61M 2205/0266; A61M 2210/1032

(57) ABSTRACT

Provided is a tracheostomy tube structure, which includes a temperature-controllable and deformable wire embedded in the wall of a flexible tube. The wire deforms in response to temperature changes, thereby altering the diameter of the tube. This allows the tracheostomy tube to be inserted with a reduced diameter, enhancing patient comfort during the procedure. After the tracheostomy tube is in place, the tube can expand to a larger diameter as the wire deforms, preventing the accumulation of sputum that could lead to biofilm formation and addressing the issue of sputum accumulation in the space between the tracheostomy tube and the trachea, thereby facilitating better breathing for the patient.

### TECHNICAL FIELD

The present disclosure relates to a tracheostomy tube structure, and more particularly to an improved tracheostomy tube structure for medical applications.

### BACKGROUND

At present, tracheostomy tubes are commonly used for patients with respiratory failure or other respiratory-related medical needs. After the patient's trachea is incised, a tracheostomy tube is inserted through the tracheal incision, so that the patient's lungs can communicate with ambient air or be connected to a ventilator, thereby maintaining airway patency and ventilation and preventing airway obstruction.

However, due to respiratory requirements, conventional tracheostomy tubes cannot have an excessively small diameter; otherwise, airflow resistance becomes excessive, thereby increasing the difficulty of breathing for the patient. Moreover, if used concurrently with a ventilator, an excessively small tube diameter may make it difficult to appropriately set the cuff pressure, thereby causing air leakage problems. On the other hand, if the diameter of the tracheostomy tube is excessively large, the tracheostomy tube is difficult to insert into the tracheostomy stoma formed after the tracheal incision, thereby complicating the placement of the tracheostomy tube. Also, subsequent compression of the trachea by the tracheostomy tube may cause further injury to the patient's respiratory system.

Furthermore, a tracheostomy tube may be used in combination with a liquid-filled or gas-filled cuff to fill the gap formed between the inserted tracheostomy tube and the patient's trachea, thereby preventing potential air leakage problems that may occur when gas in the chest is pressurized. However, although the use of a cuff can prevent air leakage, if cuff pressure is insufficient, gaps may still form between the inserted tracheostomy tube and the patient's trachea, thereby still resulting in air leakage. On the other hand, if excessively high cuff pressure is applied, the patient's trachea is likewise compressed, thereby causing pressure injury and damaging the patient's respiratory system.

Moreover, regardless of whether a cuff is used, because the inserted tracheostomy tube and the patient's trachea cannot achieve complete apposition, when the patient coughs, sputum often accumulates in the gap formed between the inserted tracheostomy tube and the patient's trachea. Especially when a cuff is used, because the cuff blocks the expulsion of sputum, sputum often enters the dead space between the tracheostomy tube and the trachea, such that the sputum cannot be expelled from the body in a timely manner, thereby resulting in biofilm formation and leading to a risk of aspiration pneumonia, which seriously affects the patient's prognosis.

Accordingly, there remains an urgent need to overcome the foregoing problems in the prior art.

### SUMMARY

In view of the foregoing deficiencies in the prior art, the present disclosure provides a tracheostomy tube structure that includes a flexible tube having a first end section and a second end section, and a temperature-controllable and deformable wire embedded in a wall of the tube.

In at least one embodiment of the present disclosure, the tube is made of an insulating material.

In at least one embodiment of the present disclosure, the tube is formed into a frustoconical shape or a tapered shape.

In at least one embodiment of the present disclosure, the wire comprises a plurality of annular structures, each annular structure having a notch, and the annular structures being arranged in a spaced and stacked configuration with respect to one another.

In at least one embodiment of the present disclosure, at least two of the plurality of annular structures have different widths.

In at least one embodiment of the present disclosure, the plurality of annular structures have widths increasing progressively from the first end section toward the second end section.

In at least one embodiment of the present disclosure, the wire further comprises a rod-shaped structure, and at least one end of the rod-shaped structure is exposed on the tube.

In at least one embodiment of the present disclosure, the plurality of annular structures are connected to one another by the rod-shaped structure.

In at least one embodiment of the present disclosure, the temperature-controllable and deformable wire is made of a shape memory alloy. In some embodiments of the present disclosure, the shape memory alloy is a nickel-titanium alloy.

In at least one embodiment, by embedding the temperature-controllable and deformable wire in the wall of the flexible tube, the tracheostomy tube structure of the present disclosure causes the wire to deform in response to temperature changes, thereby altering the diameter of the tube. As compared with the prior art, the tracheostomy tube structure of the present disclosure enables the tracheostomy tube to be placed in a reduced-diameter state, thereby improving patient comfort. After placement of the tracheostomy tube is completed, the tube can expand to a larger-diameter state as the wire deforms, thereby reducing resistance during breathing to meet the patient's respiratory requirements. Because the tracheostomy tube closely apposes the trachea, air leakage can be avoided when a ventilator is used. In addition, accumulation of sputum between the tracheostomy tube and the trachea is avoided, thereby preventing biofilm formation and reducing the risk of aspiration pneumonia.

### BRIEF DESCRIPTION OF THE DRAWINGS

The present disclosure can be more fully understood by reading the following description of the embodiments and by reference to the accompanying drawings.

FIG. 1A is a top perspective schematic view of the tracheostomy tube structure of the present disclosure.

FIG. 1B is a side perspective schematic view of the tracheostomy tube structure of the present disclosure from one viewpoint.

FIG. 1C is a side perspective schematic view of the tracheostomy tube structure of the present disclosure from another viewpoint.

FIGS. 2A and 2B are partial top schematic views of the tracheostomy tube structure of the present disclosure at different temperatures.

FIGS. 3A and 3B are side schematic views of the tracheostomy tube structure of the present disclosure in an application example.

### DETAILED DESCRIPTION

The embodiments of the present disclosure are described below by way of examples. A person having ordinary skill in the art can readily understand other advantages and effects of the present disclosure from the disclosures set forth herein.

It should be understood that the structures, proportions, and dimensions illustrated in the accompanying drawings are provided solely to facilitate understanding and reading of the disclosures set forth in the specification by a person having ordinary skill in the art and are not intended to limit the conditions under which the present disclosure can be implemented. Accordingly, any modification of structures, changes in proportional relationships, or adjustments in sizes that do not affect the effects achievable by the present disclosure or the objectives attainable thereby should be deemed to fall within the scope of the technical content disclosed herein.

As used herein, the terms “upper,” “lower,” “inner,” “outer,” “front,” and “rear” are provided for convenience of description only and are not intended to limit the scope within which the present disclosure may be implemented. Any change or adjustment in their relative relationships, without substantially altering the technical content, shall be regarded as within the implementable scope of the present disclosure.

As used herein, the articles “a” and “an” are employed to describe elements and components disclosed herein. This usage is for convenience of description and to provide a general meaning to the scope of the present disclosure. Accordingly, unless clearly indicated otherwise, such description should be understood to include one or at least one, and the singular also encompasses the plural.

As used herein, the terms “first,” “second,” and similar ordinals are primarily employed to distinguish or reference identical or similar elements or structures and do not necessarily imply any spatial or temporal order. It should be understood that, in certain circumstances or embodiments, such ordinals may be used interchangeably without affecting the implementation of the present disclosure.

In addition, as used herein, the terms “comprise,” “include,” “have,” and any other similar terminology are intended to encompass non-exclusive inclusion. For example, an element or structure that comprises a plurality of constituents is not limited to only those constituents expressly listed herein but may include other constituents that are not expressly listed yet are ordinarily inherent to such element or structure.

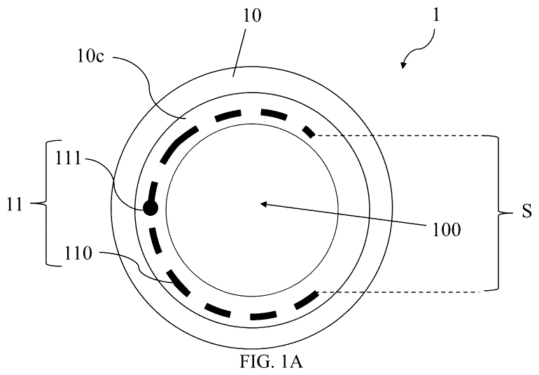

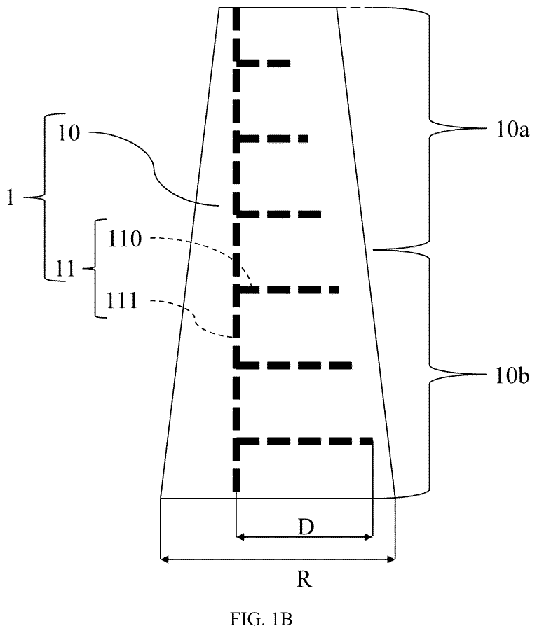

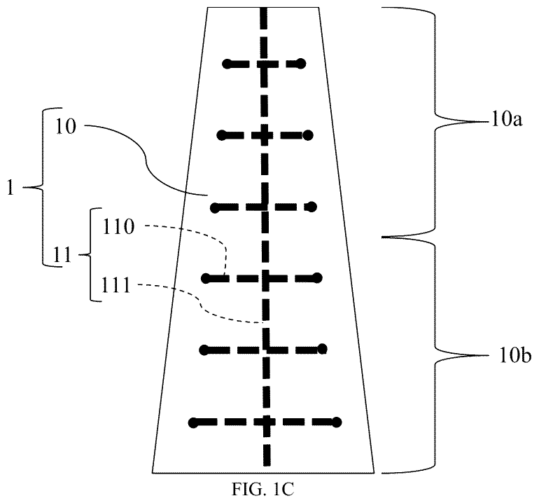

As shown in FIGS. 1A to 1C, a top perspective schematic view and side perspective schematic views taken from different viewing angles of the tracheostomy tube structure 1 are respectively illustrated. In at least one embodiment, the tracheostomy tube structure 1 of the present disclosure includes a flexible tube 10 and a temperature-controllable and deformable wire 11 encased by the flexible tube 10.

In at least one embodiment, the tube 10 of the present disclosure is made of an insulating material, such as polyvinyl chloride (PVC), polyurethane (PU), or silicone, but is not limited thereto. In some embodiments of the present disclosure, the tube 10 is formed of a biocompatible material and is therefore suitable for use as an invasive medical device.

As shown in FIG. 1B, in at least one embodiment of the present disclosure, the tube 10 has opposing first end section 10a and second end section 10b. As shown in FIGS. 1A and 1B, in some embodiments of the present disclosure, the width R of the lumen 100 of the tube 10 increases progressively from the first end section 10a toward the second end section 10b, such that the tube 10 is formed into a frustoconical shape or a tapered shape.

As shown in FIG. 1A, in at least one embodiment of the present disclosure, the wire 11 comprises a plurality of annular structures 110 each having a notch S configured as, for example, C-shaped rings or arc-shaped rings. As shown in FIG. 1B, the plurality of annular structures 110 are arranged in a spaced and stacked configuration from the first end section 10a toward the second end section 10b, such that the widths D of the annular structures 110 vary in size in correspondence with the width R of the lumen 100. As shown in FIG. 1C, in some embodiments of the present disclosure, the widths D of the plurality of annular structures 110 increase progressively from the first end section 10a toward the second end section 10b.

In at least one embodiment of the present disclosure, the wire 11 comprises a rod-shaped structure 111 that connects the plurality of annular structures 110. In some embodiments of the present disclosure, the rod-shaped structure 111 connects the plurality of annular structures 110 in series, for example, at a position near the center of the arc of each annular structure 110, thereby forming a keel-shaped wire 11.

As further shown in FIG. 1A, in at least one embodiment of the present disclosure, the wire 11 is embedded in the tube wall 10c of the tube 10 and is made of a temperature-controllable and deformable material (e.g., a thermally deformable material), such as a shape memory alloy (SMA), for example, a nickel-titanium alloy. In some embodiments of the present disclosure, the temperature-controllable and deformable material may be a non-magnetic alloy of nickel and titanium, such as nitinol, which is an alloy composed of nickel and titanium in an atomic ratio of 1:1, wherein the atomic percentage of nickel is 50% and the weight percentage is about 55%.

In at least one embodiment of the present disclosure, because the shape memory alloy is a two-phase alloy, it exhibits nonlinear superelasticity and also has good corrosion resistance. In some embodiments of the present disclosure, the nickel-titanium alloy possesses tissue characteristics similar to those of human hair, bone, and tendons and can achieve a strain ratio of about 10%. Moreover, because the relationship between its stress and the strain ratio is nonlinear, when the external force causing deformation is removed, its superelasticity enables it to recover from deformation under very small stress.

For example, when the original shape is set at a temperature above 40° C., cooling from an austenite finish (Af) temperature above 40° C. to a martensite finish (Mf) temperature below 30° C. forms martensite, and the alloy material deforms under stress. When reheated to 40° C., a reverse phase transformation occurs, thereby restoring the alloy material to its original shape.

It should be understood that the shape memory effect of nickel-titanium alloys is a phase transformation process triggered by a thermal interval. In some embodiments of the present disclosure, the phase transformation temperature range of the shape memory alloy may be selected according to practical requirements, which depends on the compositional ratio of nickel and titanium in the alloy. For example, by adjusting the weight percentage of nickel in nitinol to range from 54% to 57%, with the balance being titanium, the shape memory alloy used for the wire 11 can be designed to meet practical requirements, and by applying its phase transformation temperature range and nickel-titanium composition ratio, the design objectives of “superelasticity” or “shape memory characteristics” can be achieved.

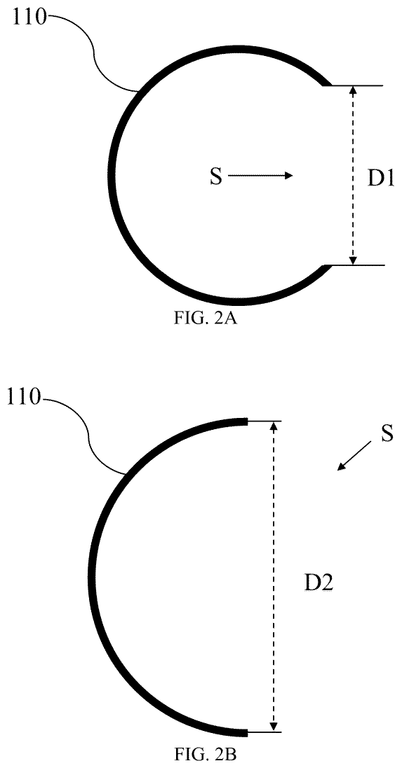

As shown in FIGS. 2A and 2B, in at least one embodiment of the present disclosure, the wire 11 may be configured such that, in a lower-temperature environment (e.g., room temperature), the annular structures 110 made of a shape memory alloy have a smaller notch S (as shown in FIG. 2A), whereas, in a higher-temperature environment (e.g., body temperature), a larger notch S is formed (as shown in FIG. 2B). Accordingly, at a first (lower) temperature (FIG. 2A), the notch width D1 is smaller than the notch width D2 at a second (higher) temperature (FIG. 2B). Therefore, when the wire 11 is encased within the tube 10 made of, for example, silicone, deformation of the wire 11 thereby alters the inner diameter of the lumen 100 of the tube 10.

In at least one embodiment of the present disclosure, the rod-shaped structure 111 of the wire 11 can serve as an electrically conductive path and is designed to be exposed at an end of the tube 10, so as to generate, via resistive heating when current passes therethrough, the temperature required to induce deformation, thereby achieving a deformation effect produced by temperature changes. It should be understood that, in some embodiments of the present disclosure, the temperature-controllable arrangement of the wire 11 may be designed according to practical requirements; that is, the magnitude of deformation produced within a given temperature interval may be tailored to practical requirements and is not particularly limited herein.

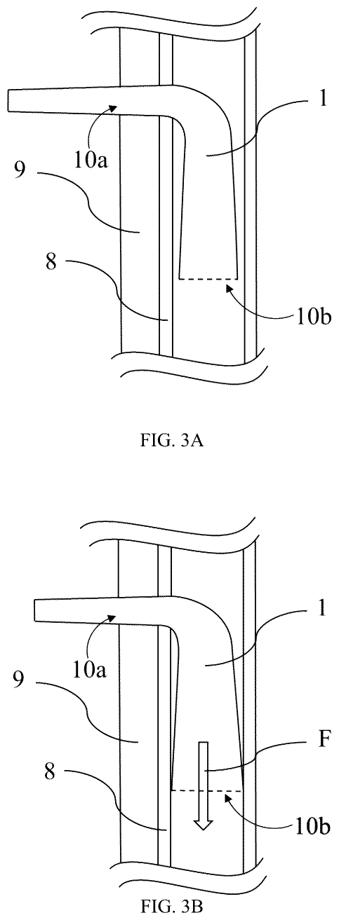

As shown in FIGS. 3A and 3B, side schematic views of the tracheostomy tube structure 1 of the present disclosure in an application example are illustrated. In at least one embodiment of the present disclosure, the first end section 10a of the tube 10 serves as a proximal segment, and the second end section 10b of the tube 10 serves as a distal segment for connection to the trachea 8.

In at least one embodiment of the present disclosure, during placement of the tracheostomy tube structure 1, because the temperature is lower (e.g., below body temperature), the annular structures 110 cause the diameter of the tube 10 to be reduced (as the annular structures 110 have a smaller notch S as shown in FIG. 2A), thereby facilitating insertion into the patient's trachea 8 at the neck 9 and improving and reducing patient discomfort during placement. In other embodiments, after the tracheostomy tube structure 1 has been placed and successfully inserted into the patient's trachea 8, the annular structures 110 within the second end section 10b, due to an increased temperature (e.g., warmed by body temperature), cause the diameter of the tube 10 to increase (as the annular structures 110 have a larger notch S as shown in FIG. 2B), so that the tube 10 closely apposes the wall of the trachea 8, and the lumen 100 available for airflow becomes larger. Also, the trachea 8 can even be slightly expanded, thereby increasing the velocity of airflow F. It should be understood that the present disclosure may also utilize the rod-shaped structure 111 of the wire 11 as an electrically conductive path, which is exposed at the first end section 10a of the tube 10, to connect to a power source and thereby produce a temperature change.

In some embodiments of the present disclosure, the wire 11 may also be connected, via its portion exposed at an end of the tube 10, to an additional temperature-control unit to regulate temperature changes of the wire 11, thereby changing the diameter of the tracheostomy tube through deformation of the shape memory alloy.

Accordingly, in some embodiments, by means of a wire 11 made of a shape memory alloy and through the arrangement of the annular structures 110, the tracheostomy tube structure 1 of the present disclosure produces a temperature-induced deformation effect. Thus, during placement, because the diameter of the tube 10 is smaller, the difficulty of placement is effectively reduced, and at body temperature, the diameter of the tube 10 is enlarged, so that the tracheostomy tube can closely appose the wall of the trachea 8, thereby avoiding various drawbacks associated with the use of cuffs in the prior art.

In at least one embodiment, the tracheostomy tube structure 1 of the present disclosure may further include a guiding device having higher hardness to assist in placement of the tracheostomy tube. In some embodiments, the actual temperature of the device may be determined through a temperature-sensing display unit disposed on the surface of the tracheostomy tube structure 1. For example, during use, the tracheostomy tube may first be removed from a refrigerator, and, after warming to approximately 20° C., the tracheostomy tube is placed. Because the guiding device does not warm up too quickly during the placement process, it can remain at a sufficiently low temperature, thereby providing sufficient support to facilitate placement of the tracheostomy tube.

In some embodiments of the present disclosure, when removal of the tracheostomy tube is required, a low temperature may be introduced through the wire, or the temperature may be lowered via the guiding device. Owing to the intrinsic properties of the shape memory alloy, its soft plasticity can reduce the difficulty of tracheostomy tube removal and, simultaneously, during use, can substantially reduce injury to tracheal tissues caused by the tracheostomy tube, thereby decreasing the risk of conditions such as bronchomalacia. Furthermore, because of the temperature-controllable characteristics of the tracheostomy tube, the contact pressure between the tracheostomy tube and the trachea can be adjusted through temperature control during patient's use, thereby avoiding compression of the trachea by the tracheostomy tube and preventing further injury to the patient's respiratory system.

In some embodiments of the present disclosure, by means of the gradually expanding tracheostomy tube structure 1, close apposition can be effectively achieved between the second end section 10b of the tube 10 (i.e., the distal segment) and the trachea 8. Accordingly, as compared with the prior art, the tracheostomy tube structure 1 of the present disclosure can avoid the problem in which, during coughing, sputum enters the dead space and cannot be cleared that result in accumulation. In addition, because the shape memory alloy has soft and deformable characteristics, it is applicable to various tracheal morphologies (for example, the trachea itself may exhibit various stenotic variations or bronchomalacia). Also, it can reduce complexity during removal of the tracheostomy tube, while simultaneously reducing compression on surrounding tissues.

In some embodiments of the present disclosure, the support provided by the shape memory alloy to the tracheostomy tube reduces the strength requirements of conventional tracheostomy tubes, thereby enabling a reduction in the diameter of the tube and lowering manufacturing costs, while maintaining the relative strength of the tracheostomy tube.

In some embodiments of the present disclosure, the tracheostomy tube structure 1 employs a wire 11 made of a recyclable shape memory alloy, thereby effectively mitigating the problem of medical waste.

At least based on the foregoing description, the tracheostomy tube structure of the present disclosure adjusts the inner diameter of the tube by embedding a shape memory alloy in a wall of the tube and controlling temperature changes. In this manner, the tracheostomy tube can be placed in the patient in a reduced diameter state to improve patient comfort, and, after placement is completed, the tube deforms to a larger diameter state to reduce resistance during breathing and meet the patient's respiratory requirements. In addition, because the inserted tracheostomy tube closely apposes the trachea, the natural orifice is effectively enlarged, thereby preventing air leakage when a ventilator is used.

Furthermore, because the tube can closely appose the trachea and has automatic dynamic adjustability to accommodate various user-specific bronchial configurations that cause narrowing, sputum can be directly expelled during patient coughing, thereby preventing accumulation in the dead space between the inserted tracheostomy tube and the patient's trachea that would otherwise lead to biofilm formation. This can substantially reduce the risk of aspiration pneumonia and thereby improve patient prognosis.

The foregoing embodiments are provided solely to illustrate the principles and effects of the present disclosure and are not intended to limit the content thereof. Although at least one exemplary embodiment has been presented in the foregoing description, it should be understood that numerous variations of the present disclosure are still possible. Likewise, it should be understood that the embodiments described herein are not intended to limit, in any manner, the scope, uses, or aspects of the claimed subject matter. Rather, the foregoing description provides those having ordinary skill in the art with a convenient guide for implementing one or more of the described embodiments. Accordingly, the scope of protection of the present disclosure shall be as set forth in the claims that follow.

What is claimed is:

1. A tracheostomy tube structure comprising:
a flexible tube having a first end section and a second end section; and
a temperature-controllable and deformable wire embedded in a wall of the flexible tube.

2. The tracheostomy tube structure of claim 1, wherein the flexible tube is made of an insulating material.

3. The tracheostomy tube structure of claim 1, wherein the flexible tube is formed into a frustoconical shape or a tapered shape.

4. The tracheostomy tube structure of claim 1, wherein the temperature-controllable and deformable wire comprises a plurality of annular structures, each annular structure having a notch, and the annular structures being arranged in a spaced and stacked configuration with respect to one another.

5. The tracheostomy tube structure of claim 4, wherein at least two of the plurality of annular structures have different widths.

6. The tracheostomy tube structure of claim 4, wherein the plurality of annular structures have widths increasing progressively from the first end section toward the second end section.

7. The tracheostomy tube structure of claim 4, wherein the temperature-controllable and deformable wire further comprises a rod-shaped structure, and at least one end of the rod-shaped structure is exposed on the flexible tube.

8. The tracheostomy tube structure of claim 7, wherein the plurality of annular structures are connected to one another by the rod-shaped structure.

9. The tracheostomy tube structure of claim 1, wherein the temperature-controllable and deformable wire is made of a shape memory alloy.

10. The tracheostomy tube structure of claim 9, wherein the shape memory alloy is a nickel-titanium alloy.
